(19) United States
(12) Patent Application Publication
(10) Pub. No.: US 2004/0115142 A1
(43) Pub. Date: Jun. 17, 2004

(54) Compositions for industrial applications

(75) Inventors: Bob E. Sherwood, Amenia, NY (US); Joseph A. Zeleznik, Poughkeepsie, NY (US); Michael T. Mallon, New Fairfield, CT (US)

Correspondence Address: DAVIDSON, DAVIDSON & KAPPEL, LLC, 485 SEVENTH AVENUE, 14TH FLOOR, NEW YORK, NY, 10018, US

(73) Assignee: JRS Pharma LP, Patterson, NY

(21) Appl. No.: 10/655,803

(22) Filed: Sep. 5, 2003

Related U.S. Application Data
(60) Provisional application No. 60/408,259, filed on Sep. 5, 2002.

(51) Int. Cl. A61K 7/42; A61K 7/021

(52) U.S. Cl. 424/59; 424/63; 106/205.9

(57) ABSTRACT

A composition has a particulate agglomerate of microcrystalline cellulose co-processed with additive materials, such as silicon dioxide, that are in intimate association with each other, the additive materials being integrated with or at least partially coating the microcrystalline cellulose. The additive materials are present in an amount of about 1% to about 99% by weight, based on the weight of the microcrystalline cellulose. When the material is silicon dioxide, the silicon dioxide is present in an amount of about 21% to about 99% by weight. A method of preparing a composition includes forming an aqueous slurry containing a mixture of microcrystalline cellulose in the form of a wet cake and additive materials; and drying the slurry to obtain a composition comprising a plurality of agglomerated particles of microcrystalline cellulose in intimate association with the additive material. In another embodiment, the composition has a particulate agglomerate of pure additive material, such as silicon dioxide.

### BACKGROUND OF THE INVENTION

The present invention relates to a novel composition for use in various industrial applications, products and processes and to the manufacture of such novel compositions, and, in particular, to a novel composition containing silicified microcrystalline cellulose and other similar co-processed products.

Cellulose is one of the most abundant materials in the natural world. It is used as a structural material primarily by plants and some animals. The abundance of this material has led to a large body of research into its mechanical properties. Most emphasis has been placed on understanding the behavior of plant celluloses and regenerated celluloses, which are solvent spun from plant celluloses. The structure of cellulose is that of a branched linear polymer with a high degree of hydrogen bonding.

One form of cellulose, microcrystalline cellulose, is a purified, partially de-polymerized cellulose. The crystal structure of microcrystalline cellulose is that of typical cellulose with a parallel conformation of two anyhydroglucose units joined by a single β-D glycosidic linkage. Such a structure is particularly helpful when it is necessary for the material to be compressed along with other pharmaceutical compositions into a dosage form while still possessing sufficient free-flowing, lubrication, and cohesive properties to ensure that the solid dosage form remains intact after compression. Due to its inherent compressibility characteristics, microcrystalline cellulose has been utilized extensively in the pharmaceutical industry in the production of pharmaceutical tablets as a direct compression vehicle for solid dosage forms, i.e., as a binding material and to enhance compactibility. It is commercially available under the trade name EMCOCEL® from Edward Mendell Co., Inc. and as Avicel® from FMC Corp. Compared to other directly compressible excipients, microcrystalline cellulose is generally considered to exhibit superior compressibility and disintegration properties.

For various reasons, however, the wet granulation process of manufacturing pharmaceutical tablets is more desirable than the direct compression process. Unfortunately, the exposure of the microcrystalline cellulose to moisture in the wet granulation process severely reduces the compressibility of these excipients, thereby reducing the effectiveness of the microcrystalline cellulose. This loss of compressibility of microcrystalline cellulose when exposed to wet granulation had long been considered a problem in the art.

In one solution, as disclosed for example in U.S. Pat. Nos. 5,585,115, 5,725,883, 5,725,884 and 5,866,166, an excipient composition, or an aqueous slurry useful in the preparation thereof, comprises a particulate agglomerate of microcrystalline cellulose co-processed with from about 0.1% to about 20% by weight of a compressibility augmenting agent, typically silicon dioxide, where the microcrystalline cellulose and the silicon dioxide are in intimate association with each other. In some instances, the silicon dioxide is preferably colloidal silicon dioxide, which refers to extremely small silica particles, in one embodiment having an average particle size from about 1 nm to about 100 μm. As these references show, silicon dioxide, as a compressibility augmenting agent, is capable of restricting the close approach of cellulose surfaces to each other either by physically preventing these surfaces from approaching each other or by changing the environment between these surfaces from an environment that tends to promote surface-to-surface interactions (such as hydrogen-bonding) to an environment that tends to inhibit such surface-to-surface interactions between surfaces of the microcrystalline cellulose. Such silicified microcrystalline cellulose is marketed under the trade name PROSOLV® by Penwest Pharmaceuticals Co.

Silicified microcrystalline cellulose has the advantages of the following valuable properties: powder blend densification, extremely high compactibility/forming properties, thermal insulative properties, electrical insulative properties, inherent lubricity, glidant effects, releasing/non-sticking properties, enhanced compaction in combination with other excipients, and great dilution potential, even when used in wet processes. Thus far, however, commercial use of such silicified microcrystalline cellulose products has been limited to the pharmaceutical industry, in particular for use in pharmaceutical manufacturing.

Yet, it is well known that silica, in its many forms and compositions, has a multitude of industrial applications. Silica is a noncombustible, white, tasteless material that occurs naturally in the crystalline and amorphous forms, and is known to occur in seventeen crystalline phases or modes and five amorphous phases. Among the forms or compositions of silica are silica dioxide, silicon dioxide, crystalline silica, quartz, amorphous fumed silica, food grade silica, hydrophobic fumed silica, treated fumed silica, untreated fumed silica, amorphous fused silica, precipitated amorphous silica, microcrystalline silica, flint, sand (e.g., foundry sand, utility sand, fracturing sand, silica sand, glass sand, melting sand, engine sand, blasting sand, traction sand, hydraulic sands and filter sand), soft silica, condensed silica fume, cristobalite, tridymite, synthetic fused silica, hydrated precipitated silica, colloidal silica, silica dispersion, and silica aerogels. Some typical applications of silica include filler for paints, plastic, epoxy, glassmaking, papers, seed germination, cosmetics, automotive, storage, wood and metal primer, water retardation, powder coating, structural concrete forms, masonry treatment, chromatographic column treatment, filtration, wood treatment and powder metal molding, as well as packaging as jars, pails, drums, multiply paper bags, bulk bags and fiberboard containers. Fumed silicas are also useful as polishing agents in the electronics industry and offer benefits such as corrosion inhibition, adhesion and scratch resistance, as well as reinforcers of silicone compounds and sealants.

Because of the wide industrial applicability of silica, it is desirable to provide for silicified microcrystalline cellulose additional applications beyond those in the pharmaceutical tablet manufacturing field.

It is also desirable to provide microcrystalline cellulose as a carrier for silica, such as in the form of silicon dioxide, and as a carrier for materials other than silica.

It is further desirable to provide additional industrial applications for microcrystalline cellulose as a carrier for materials other than silica.

It is desirable to bring the advantages of silicified microcrystalline cellulose and related co-processed microcrystalline cellulose materials to use in other industrial applications.

### SUMMARY OF THE INVENTION

It is an object of the present invention to provide a composition of silicified microcrystalline cellulose that contains higher levels of silica than was heretofore known and that is useful in a variety of industrial applications.

It is another object of the present invention to provide a composition wherein microcrystalline cellulose is used in composition with, or as a carrier for, silica or materials in place of or in addition to silica, which composition is useful in a variety of industrial applications.

It is a further object of the present invention to provide a composition of microcrystalline cellulose with an additive material or agent, which composition is useful in fields such as coating/pigments, cosmetics/sunscreen, insulating additive for wire/cable, ceramics for insulators and computers, spark plugs, colored paper, pet foods/animal feed to supply, silica, color, minerals, etc., paint, adhesives, electroplating, carbon black in cement/mortar to prevent color leaching and carbon black floating, catalytic converters, and electronic adhesives.

In accordance with the above objects and others that will be obvious to those skilled in the art, the present invention is directed to a composition comprising a particulate agglomerate of co-processed microcrystalline cellulose and an additive material or agent.

Microcrystalline cellulose can be prepared by partially depolymerizing cellulose obtained as a pulp from fibrous plant material with mineral acid solutions or with basic hydrolysis. Hydrocellulose obtained is purified via filtration, and an aqueous slurry of the hydrocellulose is spray dried to form dry, white odorless, tasteless crystalline powder of porous particles of various sizes. Alternatively, cellulose can be subjected to the hydrolytic action of hydrochloric acid at boiling temperatures so that amorphous cellulosic material can be removed and aggregates of crystalline cellulose are formed. The aggregates are collected by filtration, neutralized, such as washing with water and aqueous ammonia, and disintegrated into small fragments, often called cellulose crystallites, by vigorous mechanical means such as a blender.

Microcrystalline cellulose is commercially available as a particulate grade, varying in average particle size from about 1 μm to about 250 μm, and as a colloidal grade, varying in average particle size from less than about 1 μm. Typically, microcrystalline cellulose has an apparent density of about 0.28 g/cm³ and a tap density of about 0.43 g/cm³.

Cellulose may be acceptable for industrial compositions discussed herein with a degree of crystallization range of approximately 85% to the low 90% range (microcrystalline cellulose), and forms of cellulose having a degree of crystallization of between that of wood pulps and that of microcrystalline cellulose are also acceptable, such as natural pulps (for example, from beet or banana pulp or cotton fibers) having a degree of crystallization of approximately 60% as well as powdered pulps having a degree of crystallization higher between that of natural pulps and that of typical microcrystalline cellulose.

Celluloses that are acceptable for many industrial compositions herein also include cellulose with degrees of polymerization that are higher or lower than that of typical microcrystalline cellulose. Whereas microcrystalline cellulose obtained through acid hydrolysis typically has a degree of polymerization of generally less than approximately 350 DP, cellulose ethers, which typically have a degree of polymerization of approximately 50 DP, powdered celluloses, which typically have degrees of polymerization of from about 600 DP to about 800 DP, and certain pulps having a degree of polymerization of between about 1200 DP and about 2000 DP are also acceptable. Thus, forms of cellulose having a degree of polymerization of between about 50 DP and about 3000 DP are acceptable, although cellulose having a degree of polymerization of between about 100 DP and about 800 DP is preferable, and cellulose having a degree of polymerization of between about 150 DP and about 350 DP is even more preferable.

In one preferred embodiment, the additive material or agent comprises silica, such as silicon derivatives, including silicas and silicates, preferably silicon dioxide, and in particular colloidal silicon dioxide. Other preferable types of silica additive materials comprise aluminum silicate (Al₂O₃.4SiO₂.H₂O); hydrated or purified magnesium aluminum silicates, such as Polargel, Veegum, Montmorillonite or Bentonite, as manufactured by American Colloid Company and R.T. Vanderbilt Company, Inc., among others; and precipitated, amorphous silica (H₂SiO₃), also known as Syloid®. Other such preferred additive materials include zirconium dioxide (ZrO₂), aluminum oxide (Al₂O₃), titanium dioxide (TiO₂), dibasic calcium phosphate (DCP, CaHPO₄.2H₂O or CaHPO₄), calcium sulfate (Ca(SO₄)), calcium carbonate (CaCO₃), polyvinyl pyrollidone (PVP), carbon black, sodium lauryl sulfate (SLS, C₁₂H₂₅SO₄Na), barium sulfate (BaSO₄) and other materials. More than one such additive material may be co-processed with microcrystalline cellulose.

The relative amount of silicon dioxide or other additive material co-processed with the microcrystalline cellulose is dependent, in part, upon the type of silicon dioxide or other additive material selected. For purposes of the present invention, the amount of additive material is generally described as an effective amount, i.e., an amount that enhances or augments the specific industrial purpose for which it is desired. The microcrystalline cellulose and the additive material are preferably in intimate association with each other, and the ratio of microcrystalline cellulose to additive material is from about 1:99 to about 99:1 by weight, depending upon the requirements of the composition's use. When silicon dioxide is used as the additive material, the microcrystalline cellulose and silicon dioxide are preferably in intimate association with each other, and the composition can be prepared with a ratio of microcrystalline cellulose to silicon dioxide of from about 99:1 to about 1:99 by weight. In one embodiment, silicon dioxide is present in the composition in amounts ranging from about 21% to about 99% by weight, based on the amount of microcrystalline cellulose. The silicon dioxide or other additive material portion of the agglomerate may be in the form of an aqueous solution or slurry prior to being co-processed with hydrolyzed cellulose.

The present invention is further directed to an agglomerated composition that is derived from an aqueous slurry. The slurry, which includes hydrocellulose, hydrolyzed cellulose or microcrystalline cellulose in the form of a wet cake, at least one additive, such as silicon dioxide or another material or agent, and other optional ingredients, is dried in a manner that inhibits the formation of hydrogen bonds in the microcrystalline cellulose (intra-molecular and/or inter-molecular bonding).

The present invention is further directed to an aqueous slurry useful in the preparation of a composition useful in various industrial applications and methods, comprising a mixture of hydrocellulose, hydrolyzed cellulose or microcrystalline cellulose in the form of a wet cake and at least one additive, such as silicon dioxide or other materials. The solids content of the aqueous slurry is from about 0.5% to about 40% by weight.

Silicon dioxide or other additive materials that create physical barriers between microcrystalline cellulose surfaces include silicon dioxide or other additive materials having a very fine particle size, e.g., from about 1 nm to about 100 μm. A most preferred silicon dioxide is colloidal silicon dioxide. In certain preferred embodiments, other additive materials, such as those named above, may have at least some physical characteristics similar to those of silicon dioxide.

In one embodiment of the invention, a microcrystalline cellulose carrier composition is prepared by adding silica, preferably silicon dioxide, more preferably colloidal silicon dioxide, to microcrystalline cellulose in the form of a wet cake on a weight:weight basis. In another embodiment of the invention, a microcrystalline cellulose carrier composition is prepared by adding an additive material or agent, such as one of those named above other than silica, to microcrystalline cellulose in the form of a wet cake on a weight:weight basis. The quantity of silicon dioxide or other additive material that is added is determined based on the desired percentage of silicon dioxide or other additive material in the dry mass of microcrystalline cellulose and silicon dioxide or other additive material in the final product. Silicon dioxide or other additive material (dry, wetted or dispersed in water or other liquids) is added to microcrystalline cellulose in the form of a wet cake in a way such that the silicon dioxide or other additive material is thoroughly distributed throughout the suspension by means of mechanical agitation. In some cases, the additive may be added through in situ precipitations (e.g., the additive is formed in the slurry through a chemical reaction).

The microcrystalline cellulose/additive material composition can then be spray dried for industrial applications. The materials are co-spray dried by introducing the suspension into the drier in a way that causes the suspension to be atomized (small droplets similar to an aerosol are formed) by mechanical means, such as rotary atomization or spray nozzle atomization. In preferred embodiments of the present invention, the aqueous slurry of the microcrystalline cellulose in the form of a wet cake and additive material is introduced into the spray dryer as a single aqueous medium. However, it is possible to introduce each ingredient into separate aqueous media that are then combined in the drier. Alternatively, at least one of the substances, preferably the silica or other additive material, may be added to the drier, separately and in a dry form.

The drier is heated to an extent that facilitates solvent evaporation leaving a dry powder, which is collected. Drier temperature may be varied to change evaporative properties, changing the final product physical and functional characteristics. Similarly, other factors, such as the suspension fluid flow rate, the atomizer wheel speed, the rotary spray nozzle speed and the nozzle head pressure, may be adjusted to achieve the desired physical and functional characteristics of the final product.

In a further embodiment, the relative amount of silicon dioxide or other additive material that is co-processed with the microcrystalline cellulose can be increased to 100%, such that the composition is entirely silicon dioxide or other additive material. The method of manufacturing of the composition of this embodiment is the same as the methods discussed above, and the resulting composition comprises a particulate agglomerate of additive material, such as silica, in particular silicon dioxide, and more particularly colloidal silicon dioxide.

### DETAILED DESCRIPTION OF THE INVENTION

Compositions of the present invention comprise microcrystalline cellulose and one or more additional functional ingredients for industrial applications.

Microcrystalline cellulose is a well-known diluent, binder and disintegrant when used for pharmaceutical tablet manufacturing, and in that field its chief advantage is that it can be directly compressed into self-binding tablets that disintegrate rapidly when placed into water. The widely-used microcrystalline cellulose ingredient is prepared by partially depolymerizing cellulose obtained as a pulp from fibrous plant material with mineral acid solutions. Other forms of microcrystalline cellulose are also effective when made with basic hydrolysis. Following hydrolysis, hydrocellulose thereby obtained is purified via filtration, and an aqueous slurry of the hydrocellulose is spray dried to form dry, white odorless, tasteless crystalline powder of porous particles of various sizes. In this regard, one of ordinary skill in the art will appreciate that the terms “microcrystalline cellulose in the form of a wet cake”, “hydrocellulose”, and “hydrolyzed cellulose” are synonymous, encompass materials prepared by partially depolymerizing cellulose obtained as a pulp, and are the precursor of the (dried) microcrystalline cellulose product. These terms are also deemed to encompass processed microcrystalline cellulose, such as EMCOCEL®, that has been resuspended or rehydrated in an aqueous slurry.

Another method of preparing microcrystalline cellulose is disclosed in U.S. Pat. No. 3,141,875, which discloses subjecting cellulose to the hydrolytic action of hydrochloric acid at boiling temperatures so that amorphous cellulosic material can be removed and aggregates of crystalline cellulose are formed. The aggregates are collected by filtration, washed with water and aqueous ammonia and disintegrated into small fragments, often called cellulose crystallites, by vigorous mechanical means such as a blender.

Microcrystalline cellulose is commercially available in types within two major particle size ranges. One type of microcrystalline cellulose is a particulate grade, varying in average particle size from about 1 μm to about 250 μm. The other type of microcrystalline cellulose is a colloidal grade, varying in average particle size from less than about 1 μm, and which often by definition must have 80% of the particles below 0.20 μm. Different industrial applications will be better served by one type or the other, and the present invention is deemed to cover both types of microcrystalline cellulose.

Microcrystalline cellulose is water-insoluble. Typically, microcrystalline cellulose has an apparent density of about 0.28 g/cm³ and a tap density of about 0.43 g/cm³. See Handbook of Pharmaceutical Excipients, © 1986 American Pharmaceutical Association, pages 53-55.

The term “microcrystalline cellulose” as used in this application also includes other forms of cellulose that are acceptable for many industrial compositions, including those forms of cellulose with degrees of crystallization that are lower than that of typical microcrystalline cellulose. Whereas wood pulps may be considered to be too crude for many applications herein, microcrystalline cellulose typically has a degree of crystallization of approximately 85% and often even higher, into the low 90% range. Thus, forms of cellulose having a degree of crystallization of between that of wood pulps and that of microcrystalline cellulose are also acceptable, such as natural pulps (for example, from beet or banana pulp) having a degree of crystallization of approximately 60% as well as powdered pulps having a degree of crystallization higher between that of natural pulps and that of typical microcrystalline cellulose.

Similarly, the term “microcrystalline cellulose” as used in this application also includes other forms of cellulose that are acceptable for many industrial compositions, including those forms of cellulose with degrees of polymerization that are higher or lower than that of typical microcrystalline cellulose. Microcrystalline cellulose obtained through acid hydrolysis typically has a degree of polymerization of generally less than approximately 350 DP. Powdered celluloses, which typically have degrees of polymerization of from about 600 DP to about 800 DP, are acceptable for industrial applications described herein, as are certain pulps having a degree of polymerization of between about 1200 DP and about 2000 DP. In addition, cellulose ethers, which typically have a degree of polymerization of approximately 50 DP, are also acceptable. Thus, forms of cellulose having a degree of polymerization of between about 50 DP and about 3000 DP are acceptable, although forms of cellulose having a degree of polymerization of between about 100 DP and about 800 DP are preferable, and forms of cellulose having a degree of polymerization of between about 150 DP and about 350 DP are even more preferable.

Celluloses for this application also include derivatized celluloses, such as carboxymethyl cellulose, acetyl cellulose, hydroxypropyl cellulose, ethyl cellulose and the like. Similarly, celluloses for this application also include sulfonated and phosphorylated celluloses.

When utilized in industrial applications, microcrystalline cellulose is typically used in amounts of 1% of the formulation or more. However, it is known to use more or less microcrystalline cellulose, depending upon the requirements of the industrial composition's use.

The present invention is directed in part to a novel agglomerated microcrystalline cellulose composition that comprises a combination of microcrystalline cellulose as described above together in intimate association with certain additive materials or agents. The novel agglomerated microcrystalline cellulose composition is prepared in a manner that significantly reduces the hydrogen bonding between inter- and/or intra-molecular cellulose-to-cellulose bonding that occurs when regular, commercial grade microcrystalline cellulose is exposed to moisture (water). This can be accomplished, e.g., by preparing an aqueous slurry of microcrystalline cellulose in the form of a wet cake (i.e., hydrocellulose or hydrolyzed cellulose), one or more additive materials and other optional ingredients, and drying the mixture in a manner which inhibits quasi-homification.

The novel agglomerated augmented microcrystalline cellulose compositions of the present invention include an amount of one or more additive materials that (i) physically restrict the proximity of the interface between adjacent cellulose surfaces; (ii) inhibit interactions between adjacent cellulose surfaces, for example, via the creation of a hydrophobic boundary at cellulose surfaces; or (iii) accomplish both (i) and (ii) above.

When the composition is utilized in industrial applications, the additive materials or agents are present in the novel compositions in amounts ranging from about 1% to about 99% by weight of microcrystalline cellulose, depending upon the requirements of the composition's use. When the additive materials or agents are silica based, the additive materials are preferably present in the novel compositions in amounts ranging from about 21% to about 99% by weight of microcrystalline cellulose.

In one preferred embodiment of the invention, the additive material or agent comprises silica, such as silicon derivatives, including silicas and silicates. In another more preferred embodiment, the additive material is silicon dioxide, in particular colloidal silicon dioxide. Other preferable types of silica additive materials comprise aluminum silicate (Al₂O₃.4SiO₂.H₂O); hydrated or purified magnesium aluminum silicates, such as Polargel, Veegum, Montmorillonite or Bentonite, as manufactured by American Colloid Company and R.T. Vanderbilt Company, Inc., among others; and precipitated, amorphous silica (H₂SiO₃), also known as Syloid®, manufactured by W.R. Grace & Co., Davison Chemical Division. In these embodiments, the agglomerated microcrystalline cellulose composition may be known as a silicified microcrystalline cellulose composition.

In other preferred embodiments of the invention, the additive material that provides a physical barrier between adjacent cellulose surfaces can be materials including zirconium dioxide (ZrO₂), aluminum oxide (Al₂O₃), titanium dioxide (TiO₂), dibasic calcium phosphate (DCP, CaHPO₄.2H₂O or CaHPO₄), calcium sulfate (Ca(SO₄)), polyvinyl pyrollidone (PVP), carbon black, sodium lauryl sulfate (SLS, C₁₂H₂₅SO₄Na), barium sulfate (BaSO₄), and other materials. More than one such additive material may be co-processed with microcrystalline cellulose.

This invention is deemed to encompass all forms of these additive materials having an average primary particle size from about 1 nm to about 100 μm, and/or a surface area from about 10 m²/g to about 500 m²/g. It is preferred that these materials be of the fine particle size variety, preferably about 180 m²/g to about 350 m²/g, and most preferably about 200 m²/g. The pharmaceutical and industrial applications of these compositions may depend upon the fineness of the additives.

As stated previously, the preferred additive material or agent comprises silica, such as silicon derivatives, including silicas and silicates, and more preferably silicon dioxide, in particular colloidal silicon dioxide. Silicon dioxide is obtained by insolubilizing dissolved silica in sodium silicate solution and is insoluble in water. When obtained by the addition of sodium silicate to a mineral acid, the product is termed silica gel. When obtained by the destabilization of a solution of sodium silicate in such a manner as to yield very fine particles, the product is termed precipitated silica.

Those skilled in the art will appreciate that the name and/or method of preparation of the silicon dioxide utilized in the present invention is not determinative of the usefulness of the product. Rather, as previously mentioned, it has been surprisingly discovered that, in one preferred embodiment of the invention, it is the physical characteristics of the silicon dioxide that are critical for the industrial processes. In particular, it has been discovered that silicon dioxide having a relatively large particle size (and correspondingly small surface area), such as silica gel, is not useful in the preparation of the improved microcrystalline cellulose products of the invention.

The silicon dioxide utilized in the invention is preferably of the very fine particle size variety. In the more preferred embodiments of the invention, the silicon dioxide utilized is a colloidal silicon dioxide. Colloidal silicon dioxide is a submicron fumed silica prepared by the vapor-phase hydrolysis (e.g., at 1110° C.) of a silicon compound, such as silicon tetrachloride. The product itself is a submicron, fluffy, light, loose, bluish-white in color, odorless and tasteless amorphous powder that is commercially available from a number of sources, including Cabot Corporation (under the trade name Cab-O-Sil®, which is amorphous fumed silica, or Cab-O-Sperse®, which is in the form of aqueous dispersion of filmed silica pre-dispersed in water); Degussa, Inc. (under the trade name Aerosil®; E.I. DuPont & Co.; and W.R. Grace & Co. Colloidal silicon dioxide is also known as colloidal silica, fumed silica, light anhydrous silicic acid, silicic anhydride, and silicon dioxide fumed, among others. A variety of commercial grades of colloidal silicon dioxide are produced by varying the manufacturing process. These modifications do not affect the silica content, specific gravity, refractive index, color or amorphous form. However, these modifications are known to change the particle size, surface areas and bulk densities of the colloidal silicon dioxide products.

The surface area of the preferred class of silicon dioxides utilized in the invention preferably ranges from about 50 m²/gm to about 500 m²/gm, more preferably about 180 m²/g to about 350 m²/g. The average primary particle diameter of the preferred class of silicon dioxides utilized in the invention preferably ranges from about 5 nm to about 50 μm. However, in commercial colloidal silicon dioxide products, these particles are agglomerated or aggregated to varying extents. The bulk density of the preferred class of silicon dioxides utilized in the invention preferably ranges from about 20 g/l to about 100 g/l.

Commercially available colloidal silicon dioxide products have, for example, a BET surface area ranging from about 50±15 m²/gm (Aerosil OX50) to about 400±20 (Cab-O-Sil S-17) or 390±40 m ²/gm (Cab-O-Sil EH-5). Commercially available particle sizes range from a nominal particle diameter of 7 nm (e.g., Cab-O-Sil S-17 or Cab-O-Sil EH-5) to an average primary particle size of 40 nm (Aerosil OX50). The density of these products range from 72.0±8 g/l (Cab-O-Sil S-17) to 36.8 g/l (e.g., Cab-O-Sil M-5). The pH of the these products at 4% aqueous dispersion ranges from pH 3.5-4.5. These commercially available products are described for exemplification purposes of acceptable properties of the preferred class of silicon dioxides only, and this description is not meant to limit the scope of the invention in any manner whatsoever.

In other embodiments of the present invention, it has been discovered that the industrial applicability of microcrystalline cellulose that is wet granulated is significantly improved by a wider range of silicon dioxide products. Thus, in embodiments of the present invention where an improvement in overall compressibility of the microcrystalline cellulose (whether utilized in wet granulation or dry granulation) is not important, and the microcrystalline cellulose product is to be subjected to wet granulation, it has been discovered that the surface area of the silicon dioxide can be as low as about 50 m²/gm and the average primary particle diameter can be as large as about 100 μm. Such silicon dioxide products are also deemed to be encompassed within the scope of the invention.

The co-processed product comprises microcrystalline cellulose and at least one additive material or agent, such as silicon dioxide, in intimate association with each other. Magnifications of the resultant particles indicate that the additive material is integrated with, or at least partially coats, the surfaces of the microcrystalline cellulose particles. The amount of the additive material included in the composition is preferably greater than about 1% by weight relative to the microcrystalline cellulose, and the additive material appears to substantially coat the surfaces of the microcrystalline cellulose particles. When the additive material is silicon dioxide, the amount of the silicon dioxide included in the composition is preferably greater than about 20% by weight relative to the microcrystalline cellulose, and the silicon dioxide appears to substantially coat the surfaces of the microcrystalline cellulose particles. The amount of the additive material included in the composition can be as much as about 99% by weight relative to the microcrystalline cellulose. The exact relationship of the two or more ingredients of the composition after co-processing is not presently understood; however, for purposes of description the co-processed particles are described herein as including an agglomerate of microcrystalline cellulose and an additive coating material in intimate association with each other.

One skilled in the art will appreciate that other classes of materials or compounds having size, surface area and other physical characteristics similar to those of silicon dioxide may be useful in physically forming a barrier that may reduce the surface-to-surface interactions (including hydrogen-bonding) between cellulose surfaces. Such materials include (but are not limited to) non-silicon metal oxides, preferably colloidal. In certain preferred embodiments of the invention, the additive material used is a fumed metal oxide, such as zirconium dioxide (ZrO₂), aluminum oxide (Al₂O₃) and titanium dioxide (TiO₂), as well as others, prepared by methods well known in the art.

It is preferred in the present invention that the microcrystalline cellulose and additive materials or agents, such as silicon dioxide, are coprocessed, resulting in an intimate association of these ingredients, rather than being combined, e.g., as a dry mixture. In preferred embodiments of the present invention, an aqueous slurry of the microcrystalline cellulose in the form of a wet cake (i.e., hydrocellulose or hydrolyzed cellulose), the at least one additive material and other optional ingredients is prepared in order to obtain (after a drying step) agglomerated particles wherein these components are intimately associated. In preferred embodiments of the present invention, the aqueous slurry of the microcrystalline cellulose in the form of wet cake (i.e., hydrocellulose or hydrolyzed cellulose) and additive materials is introduced into the spray drier as a single aqueous medium. However, it is possible to separately introduce each ingredient into separate aqueous media that are then combined. Alternatively, at least one of the substances, most preferably the silica or other additive material, may be added to the drier, separately and in a dry form. Other procedures for combining these materials with or without other optional ingredients known to those skilled in the art are deemed to be equivalent to the preferred spray-drying technique described herein and are further deemed to be encompassed by the appended claims.

In preferred embodiments of the present invention, the coprocessing of the microcrystalline cellulose and the additive materials is accomplished by forming a well-dispersed aqueous slurry of microcrystalline cellulose in the form of a wet cake (i.e., hydrocellulose or hydrolyzed cellulose) into which the additive material has been dissolved, and thereafter drying the slurry and forming a plurality of coated or integrated, and in some embodiments silicified, microcrystalline cellulose particles. Typically, microcrystalline cellulose in the form of a wet cake (i.e., hydrocellulose or hydrolyzed cellulose) is first added to an aqueous solution so that a slurry or suspension containing from about 0.5% to about 40% hydrocellulose in the form of solids is obtained. At this stage, it is optionally desirable to adjust the pH of the slurry to about neutral with ammonium hydroxide, sodium hydroxide, and mixtures thereof or the like. The suspension is kept under constant agitation for a sufficient time to assure a uniform distribution of the solids prior to being combined with the additive materials, such as silicon dioxide. Viscosity modifiers, such as surfactants and alcohols, can be used to improve processability for certain material combinations, such as to allow more optimal slurry concentrations, and this is deemed to be encompassed by the appended claims.

In certain embodiments, the slurry is prepared from commercially available pulp (cellulose) sources. Examples of acceptable cellulose pulp are marketed under the trade names Temalfa, manufactured by Tembec Inc., Saicor manufactured by Sappi Saicor of South Africa, and HJHD manufactured by Rayonier, Inc. Others not listed here may also be acceptable. In addition, once the slurry has been prepared and the resultant microcrystalline cellulose processed, the microcrystalline cellulose may be resuspended or rehydrated and spray dried once more for this application (see Table 1, entry no. 34).

Cellulose pulp is generally supplied on rolls or sheets. Typically, the pulp is shredded or chopped up and then reacted with HCl and water at high temperatures (cellular hydrolysis), in order for the cellulose to be de-polymerized (i.e., to break down). The slurry is then washed and filtered with de-mineralized water and pH adjusted. The cellulose is then filtered in order to separate the microcrystalline cellulose from other cellulose, and alpha or beta cellulose, which is the crystalline region of the cellulose, is obtained.

The additive materials are added to the suspension or slurry in amounts ranging from about 1% to about 99% by weight, based on the amount of microcrystalline cellulose, depending upon the requirements of the composition's use. In one embodiment, when the additive material is silicon dioxide, it is added to the suspension or slurry in amounts ranging from about 21% to about 99% by weight, based on the amount of microcrystalline cellulose. The quantity of additive material that is added is determined based on the desired percentage of additive material in the dry mass of microcrystalline cellulose and additive material in the final product. Additive material is added to a microcrystalline cellulose suspension in a way such that the additive material is thoroughly distributed throughout the suspension by means of mechanical agitation.

There is no appreciable dissolution of either ingredient (microcrystalline cellulose in the form of a wet cake or the additive material, such as silicon dioxide), since both are relatively water insoluble. The microcrystalline cellulose in the form of a wet cake and the additive material, such as silicon dioxide, are well-dispersed in the slurry or suspension prior to drying and forming the novel particles.

It is preferred that the suspension be dried using spray-drying techniques. Other drying techniques, may include but are not limited to spray belt drying, flash drying, ring drying, micron drying, tray drying, vacuum drying, radio-frequency drying, and possibly microwave drying, although spray drying is preferred.

The coated microcrystalline cellulose materials can be spray dried for industrial applications. In the (preferred) spray-drying process, the aqueous dispersion of microcrystalline cellulose in the form of a wet cake (i.e., hydrocellulose or hydrolyzed cellulose) and additive materials is brought together with a sufficient volume of hot air to produce evaporation and drying of the liquid droplets. The highly dispersed slurry of microcrystalline cellulose in the form of a wet cake (i.e., hydrocellulose or hydrolyzed cellulose) and additive materials is pumpable and capable of being atomized. The materials are co-spray dried by introducing the suspension into the drier in a way that causes the suspension to be atomized (small droplets similar to an aerosol are formed) by mechanical means. In preferred embodiments of the present invention, the aqueous slurry of the microcrystalline cellulose and additive materials is introduced into the spray dryer as a single aqueous medium. However, it is possible to separately introduce each ingredient into separate aqueous media that are then combined in the drier. Alternatively, at least one of the substances, most preferably the additive material, may be added to the drier, separately and in a dry form. The drier is heated to an extent that facilitates solvent evaporation, and the suspension is sprayed into a current of warm filtered air, which supplies the heat for evaporation. The air is then exhausted with the removed moisture, leaving a dry powder that is collected. The resultant spray-dried powder particles are approximately spherical in shape and are relatively uniform in size, thereby possessing excellent flowability.

Drier temperature may be varied to change evaporative properties changing the final product physical and functional characteristics. The ratio of microcrystalline cellulose to additive material, such as silicon dioxide, can vary from 99:1 to 1:99 by weight.

In one embodiment, the ratio of microcrystalline cellulose to silicon dioxide is even less than 1:99 by weight, such that the compound is effectively pure silicon dioxide. In fact, in certain embodiments, certain selected industrial application may call for pure silicon dioxide to be used rather than the silicified microcrystalline cellulose compound. In such instances, a spray-dried form of the pure silicon dioxide may be used. In such a situation, the spray dried silicon dioxide is prepared by introducing the silicon dioxide to deionized water, or some other solvent, to form a suspension that may vary in total solids content. The material is spray dried by introducing the silicon dioxide suspension into the drier in a way that causes the suspension to be atomized (small droplets similar to an aerosol are formed) by mechanical means. The drier is heated to an extent that facilitates solvent evaporation, leaving a dry powder, which is collected. Drier temperature may be varied to change evaporative properties changing the final product physical and functional characteristics. In preferred embodiments for some applications, the silicon dioxide comprises colloidal silicon dioxide.

The co-processed product comprises microcrystalline cellulose and silicon dioxide or other additive materials in intimate association with each other. The exact relationship of the two ingredients of the composition after co-processing is not presently understood; however, for purposes of description the coprocessed particles are described herein as including an agglomerate of microcrystalline cellulose and silicon dioxide or other additive materials in intimate association with each other. By “intimate association”, it is meant that the silicon dioxide or other additive material has in some manner been integrated with the microcrystalline cellulose particles, e.g., via at least a partial coating of the microcrystalline particles, as opposed to a chemical interaction of the two ingredients. The term “intimate association”, therefore, is deemed for purposes of the present description as being synonymous with “integrated” or “united”. The co-processed particles are not necessarily uniform or homogeneous.

In a further embodiment, the relative amount of silicon dioxide and/or other additive material in the composition can be increased to 100%, such that the composition is entirely silicon dioxide and/or other additive material, with no microcrystalline cellulose. The method of manufacturing of the composition of this embodiment is the same as the methods discussed above, and the resulting composition comprises a particulate agglomerate of silica, in particular silicon dioxide, more particularly colloidal silicon dioxide. Magnifications of the resultant particles of this embodiment indicate that the processed product yields particles substantially similar to those of the embodiments wherein microcrystalline cellulose is present, except that the particles do not have a microcrystalline cellulose core.

After a uniform mixture of the ingredients is obtained in the suspension, the suspension is dried to provide, when the additive material is silicon dioxide, a plurality of silicified microcrystalline cellulose particles. When the additive material is not silicon dioxide, the suspension is dried to provide a plurality of additive-coated microcrystalline cellulose particles. When the mixture comprises 100% additive material, such as silicon dioxide, the suspension is dried to provide a plurality of silicon dioxide particles.

Depending upon the amount and type of drying, the concentration of the microcrystalline cellulose in the form of a wet cake (i.e., hydrocellulose or hydrolyzed cellulose) and silicon dioxide or other additive materials in the suspension, the flow rate of the fluid suspension being pumped, the wheel speed of the atomizer, the rotary spray nozzle speed or the nozzle head pressure, the resultant particles will have different particle sizes, densities, pH, moisture content, etc.

The particulate co-processed product of the present invention possesses desirable performance attributes that are not present when the combination of microcrystalline cellulose and additive materials, such as silicon dioxide, are combined as a dry mixture. It is believed that the beneficial result obtained by the combination of these materials is due to the fact that the materials are intimately associated with each other.

The average particle size of the agglomerated microcrystalline cellulose and agent composition of the present invention ranges from about 10 microns to about 1000 microns. Particle sizes of about 10-500 microns are preferred, particle sizes of about 30-250 microns are more preferred and particle sizes of about 40-200 microns are most preferred. It will be appreciated by those of ordinary skill in the art that the drying of the aqueous suspension results in a random size distribution of the novel composition particles being produced. For example, if spray drying techniques are used, droplet size, temperatures, agitation, dispersion, concentration, air flow, fluid flow rate, atomizer wheel speed, etc. will affect final particle size. Furthermore, it is within the scope of the invention to sort or mechanically alter the dried particles according to ranges of particle sizes depending upon end uses. The particle size of the integrated excipient is not narrowly critical, the important parameter being that the average size of the particle must permit the formation of a dried composition that has the desired characteristics for the chosen industrial application.

It should also be noted that, all other conditions being equal, it is possible that the average particle size of the agglomerated microcrystalline cellulose composition of the present invention will be decreased as the relative amount of microcrystalline cellulose in the composition is decreased relative to the additive material, in particular to silicon dioxide. Because, in general, the average particle size of the microcrystalline cellulose is much larger than the average particle size of the additive material, the agglomeration of microcrystalline cellulose affects the average particle size of the total composition comparatively much more, such that agglomeration of particles is much faster where more microcrystalline cellulose is present. Conversely, increasing the amount of microcrystalline cellulose even slightly allows the agglomeration of larger particles more quickly.

In certain embodiments of the invention, in addition to the microcrystalline cellulose and one or more additive materials discussed above, other additives known to those skilled in the art can be added to the novel composition prior to preparation of the final product. For example, if desired, any generally acceptable solute or insoluble inert filler material can be included in the final product. Such additional materials include cellulosics (such as cellulose ethers, celluloses esters, mixtures thereof, and the like), starches and starch derivatives (such as corn starch, high amylose corn starch, high amylopectin corn starch, sodium starch glycolate, hydroxylated starch, hydroxy ethylated starch, modified potato starch, mixtures thereof, and the like), polysaccharides (such as Guar gum, Acacia gum, Glucosamine, Chitin, Chitosan, microcrystalline Chitosan and Chitosan derivatives, and polydextrose), polypeptides (such as gelatin, polylysine, polyalanine and any polyamino acid), polyesters (such as polyacrylates, polyacetates, polypropionates and polybutyrates), polyalkanes, polyalkenes and polyalkynes (such as polyethylene, polypropylene, polybutene, polybutadiene and polyacetlyene), and resins (synthetic and natural, such as phenolic resins, terpenes, squalene). In particular, suitable additional materials that are acceptable in this invention are polyvinyl pyrollidone (PVP), polyvinyl pyrollidone/polyvinyl acetate copolymer (PVPA), polysorbate (PSB) and sodium lauryl sulfate (SLS), as used in the examples set forth below. These additives may be included in desired amounts which will be apparent to those skilled in the art.

Other additives that may be added include metal oxides, carbonaceous powders, silicates, vitamins, minerals, metal sulfides/sulfates/sulfites, metal arsenides, as well as any pigments and colors, such as aluminum lakes. For example, these may include sodium silicate, sodium aluminum silicate, graphite, charcoal, vitamins C, A, B, D, etc., calcium, zinc, iron salts, magnesium oxide, manganese oxide, or calcium, magnesium, iron, sodium sulfide, sulfate or arsenide.

Still further additives that may be added include cross carmellose sodium, docusate sodium (C₂₀H₃₇NaO₇S), diatomaceous earth (also called diatomaceous silica, SiO₂.nH₂O), hydrated dextrates (spray crystallized dextrose containing small amounts of starch oligosaccharides, available under the trade name Emdex®, carboxymethylcellulose, methylethylcellulose, hydroxypropylmethylcellulose (HPMC), kaolin, maltodextrin, polyethylene glycol (PEG), simethicone and stearic acid, which may be used for various medicinal or industrial purposes.

In further embodiments of the invention, more than one additive material may be used. Thus, for example, it is possible to use two or more agents that act as physical barriers (e.g., physically restricting the proximity of the interface between adjacent cellulose surfaces) or to use two or more agents that inhibit interactions between adjacent cellulose surfaces, for example, via the creation of a hydrophobic boundary at cellulose surfaces. In certain embodiments, for example as discussed in the examples below, silicon dioxide may be used with another additive material.

In certain preferred embodiments, two or more additive materials are used such that each provides an effect by different mechanisms, such as one agent that acts as a physical barrier (such as silicon dioxide) and another agent that inhibits interactions between adjacent cellulose surfaces. In such embodiments, it is preferred that all additive materials be incorporated into the aqueous slurry and dried (e.g., via spray drying) to form agglomerated particles in which the microcrystalline cellulose and two or more additive materials are in intimate association with each other. In preferred embodiments of the present invention, the aqueous slurry of the additive materials is introduced into the spray dryer as a single aqueous medium. However, it is possible to separately introduce each ingredient into separate aqueous media that are then combined in the drier. Alternatively, at least one of the substances, preferably one or more of the additive materials, may be added to the drier, separately and in a dry form. Such preferred embodiments are capable of providing a synergistically improved microcrystalline cellulose composition that has properties described above which are at least as good, and preferably improved, as compared to the properties of the novel microcrystalline cellulose compositions that include only one class of these additive materials, such as silicon dioxide.

In these embodiments wherein two or more additive materials are used, the exact relationship of the ingredients of the composition after co-processing is not presently understood; however, for purposes of description the co-processed particles are described herein as including an agglomerate of microcrystalline cellulose and two or more additive materials in intimate association with each other. By “intimate association”, it is meant that the additive materials have in some manner been integrated with the microcrystalline cellulose particles, e.g., via at least a partial coating of the microcrystalline particles, as opposed to a chemical interaction of the ingredients. The term “intimate association” is therefore deemed for purposes of the present description as being synonymous with “integrated” or “united”. The co-processed particles are not necessarily uniform or homogeneous.

In addition, in additional preferred embodiments of the invention, more than one additive material may be used based upon the function or functions of those additive materials in the end product composition. Thus, more than one additive material may be added to the composition as functional agents. In these embodiments, the additive materials may have functionality in the composition both separately and synergistically.

This invention is deemed to encompass all forms of these silicified or otherwise additive-augmented microcrystalline cellulose industrial compositions having a median particle size (diameter) from about 5 μm to about 350 μm. It is preferred that these materials be of median particle size of about 65 μm to about 350 μm, and most preferably about 120 μm to about 200 μm. The pharmaceutical and industrial applications of these compositions may depend upon the fineness of the compositions. Particle size can be measured using any known technique, including number weighted, volume weighted and mass weighted techniques, depending upon the industry.

The novel composition of the present invention can be used in conjunction with a wide variety of industrial applications. For example, the novel composition of microcrystalline cellulose and additives, such as silicon dioxide, may be useful in the fields of coating or pigments, cosmetics and sunscreen, insulating additive for wire and cable, ceramics for insulators and computers, spark plugs, colored paper, pet foods and animal feed to supply, silica, color, minerals, etc., paint, adhesives, electroplating, carbon black in cement and mortar to prevent color leaching and carbon black floating, catalytic converters and electronic adhesives.

In the field of coating and pigments, the novel composition of silicified microcrystalline cellulose may have light and infrared suppressive properties, making it particularly suited for use as an opacifier and an anti-reflective substance or an UV light inhibitor. With colloidal types of microcrystalline cellulose substrates, the novel composition of silicified microcrystalline cellulose would provide emulsion stabilization for colors and pigments.

In the field of thermal insulation, the novel composition of silicified microcrystalline cellulose may be particularly useful. The microcrystalline cellulose substrate would provide thermal insulation, and the (colloidal) silicon dioxide additive would provide flow/glidant properties to fill tight spaces, increase heat capacity, retard flammability, and reduce heat conductivity.

In the field of ceramics, microcrystalline cellulose and colloidal silicon dioxide are currently used separately as insulators, fillers, and mold release agents, etc. The novel composition of silicified microcrystalline cellulose may be useful, as the microcrystalline cellulose would serve as filler and carrier for colloidal silicon dioxide, which would increase heat capacity and reduce conductivity. This has utility, for example, in spark plug or heat shield applications. For high voltage ceramic insulators, microcrystalline cellulose could be burned out to provide air pocket insulating effects, and colloidal silicon dioxide would reduce the material conductivity. This also has utility as a heat sink, for example in integrated circuits.

In the field of colored textiles or paper, the novel composition of silicified microcrystalline cellulose may be particularly useful, as the microcrystalline cellulose would increase bonding to cellulosic papers and textile fibers. The combination of microcrystalline cellulose and colloidal silicon dioxide in the novel composition would provide fiber strength. For example, compare to Tensel™ fabrics.

In the field of catalytic converters, the novel composition of silicified microcrystalline cellulose may be particularly useful. It is known that colloidal silicon dioxide serves as a catalyst in the converter. In this composition, the microcrystalline cellulose would be a carrier for colloidal silicon dioxide and create porosity when burned out to produce a honeycomb-like, porous structure.

In the field of electronic adhesives, the novel composition of silicified microcrystalline cellulose may be particularly useful, as the colloidal silicon dioxide would reduce conductivity, while the microcrystalline cellulose would serve as the colloidal silicon dioxide carrier, provide greater adhesion, and reduce conductivity.

In the field of polishing compounds, such as for metals, glass or electronic components, the novel composition of silicified microcrystalline cellulose may be particularly useful. It is known that colloidal silicon dioxide or fumed silica is a mild to moderate abrasive for fine polishing applications. In this composition, the microcrystalline cellulose would be a particulate carrier for the abrasive colloidal silicon dioxide and also be slightly abrasive itself.

In many of these fields, the co-processed composition, namely the silicified or otherwise augmented microcrystalline cellulose, described herein will be combined or mixed with one or more additional ingredients or substances that may be necessary for the particular industrial application. For example, in the field of coating and pigments, silicified microcrystalline cellulose may be combined with colors and pigments. Similarly, for example, in the field of textiles or paper, silicified microcrystalline cellulose may be combined with fibers to increase strength. In such applications, the combination or mixture may be done by any conventional means, such as mixing, coating, etc. In some cases, it may be preferred that the co-processed composition be further co-processed with the additional substances, rather than being combined, e.g., as a dry mixture.

In one embodiment of this further co-processing, an aqueous slurry of the silicified or otherwise augmented microcrystalline cellulose and the other ingredients necessary for the particular industrial application is prepared in order to obtain (after a drying step) agglomerated particles wherein these components are intimately associated. In preferred embodiments, this aqueous slurry of the silicified or otherwise augmented microcrystalline cellulose and the other ingredients is introduced into the spray drier as a single aqueous medium. However, it is possible to separately introduce each ingredient, i.e., the silicified or otherwise augmented microcrystalline cellulose and the other ingredients, into separate aqueous media that are then combined. Alternatively, it is also preferable to introduce the silicified or otherwise augmented microcrystalline cellulose into the drier, separately and in a dry form, with the other substances either in an aqueous slurry or also in a dry form. Other procedures for combining these materials with or without other optional ingredients known to those skilled in the art are deemed to be equivalent to the preferred spray-drying technique described herein and are further deemed to be encompassed by the appended claims.

Some process improvements can also be addressed, including:

(a) measures to reduce atomizer/nozzle wear during the production of co-processed materials;

(b) applicability of solvent-based (non-aqueous) processing for industrial applications;

(c) new equipment and procedures for controlled introduction of the additives; and

(d) additive agents or techniques to reduce viscosity, achieve higher production rates and/or improve processability.

These examples are intended to demonstrate the broad scope of applicability of the invention to a wide variety of industrial applications. They are not meant to limit the scope of the invention in any way.

The examples described below set forth the preparation of various microcrystalline cellulose/additive material compositions in accordance with the claimed invention. Table 1 shows a tabulation of various examples of compositions prepared in accordance with the present invention, as described in the examples below.

TABLE 1

| | Slurry | Additive | Nominal | Additive | Nominal | Particle Size | | | | | | | |
|---|---|---|---|---|---|---|---|---|---|---|---|---|---|
| No. | Pulp | 1 | Level | 2 | Level | D₁₀ | | d₅₀ | | d₉₀ | | Moisture | ROI |
| 1. | Temalfa | ZrO₂ | 2.0% | | | 22.36 | μm | 68.15 | μm | 203.69 | μm | 4.9% | 1.9160 |
| 2. | Temalfa | Al₂O₃ | 2.0% | | | 19.15 | μm | 58.93 | μm | 1564.19 | μm | 3.8% | 1.9893 |
| 3. | Temalfa | TiO₂ | 2.0% | | | 17.71 | μm | 55.27 | μm | 130.30 | μm | 4.3% | 1.8788 |
| 4. | Temalfa | DCP | 2.0% | | | 19.46 | μm | 59.19 | μm | 136.53 | μm | 3.4% | 1.3913 |
| 5. | Temalfa | PVP | 2.0% | | | 17.92 | μm | 56.14 | μm | 132.72 | μm | 3.9% | |
| 6. | Temalfa | PVP | 5.0% | CSD | 5.0% | 9.91 | μm | 31.15 | μm | 104.45 | μm | 3.5% | |
| 7. | Temalfa | PVP | 5.0% | CSD | 5.0% | 9.19 | μm | 31.90 | μm | 104.92 | μm | 3.5% | |
| 8. | Temalfa | Polargel | 2.0% | | | 16.75 | μm | 54.02 | μm | 130.31 | μm | 5.6% | 1.9880 |
| 9. | Temalfa | VeeGum | 2.0% | | | 16.35 | μm | 51.15 | μm | 120.59 | μm | 2.7% | 1.8375 |
| 10. | Temalfa | Carbon | 2.0% | | | 17.40 | μm | 53.99 | μm | 130.64 | μm | 4.2% | |
| | | Black | | | | | | | | | | | |
| 11. | Temalfa | Ca(SO₄) | 2.0% | | | 14.14 | μm | 49.35 | μm | 124.31 | μm | 4.7% | 1.4835 |
| 12. | Temalfa | PVP | 20.0% | CSD | 5.0% | 15.96 | μm | 43.87 | μm | 109.29 | μm | 2.9% | |
| 13. | Temalfa | SLS | 0.1% | | | 14.86 | μm | 45.66 | μm | 114.05 | μm | 2.7% | |
| 14. | Temalfa | SLS | 0.3% | | | 14.12 | μm | 41.07 | μm | 101.84 | μm | 2.7% | |
| 15. | Temalfa | SLS | 0.5% | | | 12.37 | μm | 35.69 | μm | 95.18 | μm | 3.2% | |
| 16. | Temalfa | SLS | 0.7% | | | 11.47 | μm | 32.93 | μm | 88.79 | μm | 3.1% | |
| 17. | Temalfa | SLS | 0.9% | | | 10.30 | μm | 31.01 | μm | 87.38 | μm | | |
| 18. | Saicor | BaSO₄ | 2.0% | | | 12.81 | μm | 43.35 | μm | 102.42 | μm | 2.9% | |
| 19. | Saicor | BaSO₄ | 2.0% | | | 13.18 | μm | 41.51 | μm | 97.27 | μm | 4.1% | |
| 20. | Saicor | BaSO₄ | | | | 15.72 | μm | 47.79 | μm | 114.94 | μm | 3.7% | |
| 21. | Saicor | BaSO₄ | 2.0% | | | 12.06 | μm | 41.02 | μm | 95.75 | μm | 5.1% | |
| 22. | n/a | CSD | 100.0% | | | 6.96 | μm | 27.35 | μm | 104.36 | μm | | |
| 23. | n/a | CSD | 100.0% | | | 2.04 | μm | 7.67 | μm | 34.29 | μm | | |
| 24. | Saicor | CSD | 20.0% | | | 2.02 | μm | 6.15 | μm | 13.46 | μm | | |
| 25. | n/a | CSD | 100.0% | | | 2.07 | μm | 9.39 | μm | 52.06 | μm | | |
| 26. | n/a | CSD | 75.0% | SLS | 25.0% | 1.89 | μm | 8.34 | μm | 23.45 | μm | | |
| 27. | Temalfa | CSD | 40.0% | | | 4.72 | μm | 20.23 | μm | 80.76 | μm | 1.8% | |
| 28. | Temalfa | CSD | 60.0% | | | 3.20 | μm | 14.58 | μm | 64.08 | μm | 2.2% | |
| 29. | Temalfa | CSD | 80.0% | | | 2.65 | μm | 11.55 | μm | 43.80 | μm | 2.0% | |
| 30. | Temalfa | CSD | 20.0% | | | 13.69 | μm | 41.72 | μm | 117.26 | μm | | |
| 31. | Temalfa | PSB | 1.0% | | | 16.52 | μm | 51.28 | μm | 123.38 | μm | 3.0% | |
| 32. | Temalfa | PSB | 2.0% | | | 14.95 | μm | 46.81 | μm | 121.86 | μm | 2.6% | |
| 33. | Temalfa | CSD | 25.0% | | | 17.51 | μm | 57.99 | μm | 136.29 | μm | 2.5% | |
| 34. | Emcocel ® | | | | | 20.42 | μm | 66.30 | μm | 159.37 | μm | 2.9% | |
| 35. | Temalfa | SMC | 5.0% | | | 17.28 | μm | 53.95 | μm | 125.50 | μm | 2.8% | |
| 36. | Temalfa | SMC | 1.0% | | | 16.86 | μm | 52.60 | μm | 128.12 | μm | 2.9% | |
| 37. | Temalfa | SMC | 2.0% | | | 17.64 | μm | 55.51 | μm | 134.15 | μm | 4.5% | |
| 38. | Temalfa | COS | 2.0% | | | 19.07 | μm | 60.39 | μm | 134.29 | μm | 2.2% | |
| 39. | Temalfa | COS | 2.0% | | | 18.01 | μm | 52.85 | μm | 123.29 | μm | 2.2% | |
| 40. | Temalfa | COS | 2.0% | | | 18.65 | μm | 55.00 | μm | 127.51 | μm | 2.2% | |

### EXAMPLE 1

The example illustrates a method for preparing silicified microcrystalline cellulose slurry or other co-processed additive materials with microcrystalline cellulose.

The manufacturing process begins with high-purity cellulose pulp, which is supplied on rolls, and is then shredded and conveyed to a reactor. In the reactor, the pulp is partially de-polymerized by hydrolysis, and the hydrolyzed slurry is drained to a holding tank. The slurry is then washed and filtered with de-mineralized water, pH adjusted and stored in holding tanks. Prior to spray drying, the additive material is added to the microcrystalline cellulose in the form of wet cake on a weight:weight basis. The quantity of additive material that is added is determined based on the desired percentage of the additive material in the dry mass of microcrystalline cellulose and additive material in the final product. Dry additive material is added to a microcrystalline cellulose in the form of wet cake in a way such that the additive material is thoroughly distributed throughout the slurry by means of mechanical agitation.

The method for calculating the mass of the additive material is the same for all levels of additive material. Additionally, it may be necessary at time to adjust the slurry solids content. Calculation for the required water necessary to reduce the overall slurry solids content requires knowledge of the initial slurry solids content in addition to the targeted batch size and additive level. The table below provides a sample scheme for producing such a material, with the mathematical equations being used to calculate the required materials based on known parameters.

| | |
|---|---|
| Target batch size (B) | 10 kg |
| Desired CSD level (A) | 5.0% |
| Initial MCC slurry solids content (Sᵢ) | 20.0% |
| CSD mass required (M_{A}) | 500 g (0.5 kg) |
| MCC slurry mass required (M_{S}_{ᵢ}) | 47.5 kg |
| Target slurry solids content (S_{f}) | 15.0% |
| Additional H₂O required (M_{H}_{₂}_{O}) | 18.67 kg |

In order to determine the required additive mass, the following equation is used.

M_{A}=B×A

In order to determine the required slurry mass using slurry at initial solids content, the following equation is used.

$${M}_{{S}_{i}} = \frac{B - {M}_{A}}{{S}_{i}}$$

In order to determine the required water needed to adjust the solids content, the following equation is used. Care should be taken not to confuse Sᵢ (initial solids content) with S_{f} (final or target slurry solids content).

$${M}_{{H}_{2} O} = \frac{B}{{S}_{f}} - \frac{B - {M}_{A}}{{S}_{i}} - {M}_{A}$$

The microcrystalline cellulose in the form of wet cake is then dried in a spray dryer. The material is spray dried by introducing the suspension into the drier in a way that causes the suspension to be atomized (small droplets similar to an aerosol are formed) by mechanical means. The drier is heated to an extent that facilitates solvent evaporation leaving a dry powder, which is then collected. Drier temperature may be varied to change evaporative properties, changing the final product physical and functional characteristics.

This same process is used regardless of the additive used in combination with microcrystalline cellulose. The system is not limited to binary combinations, such that more than one additive may be added to the microcrystalline cellulose in the form of wet cake, and in differing ratios.

### EXAMPLE 2

In this example, the data from entry no. 24 in Table 1 was used. Saicor type pulp was used to form the slurry from which the microcrystalline cellulose in the form of wet cake was produced. The additive material was colloidal silicon dioxide, which was added to the microcrystalline cellulose in the form of wet cake in an amount of 20.0% weight:weight percent. After spray drying of the suspension, certain particle size resulted, of which the particle size diameter was 2.02 μm at the 10^{th} percentile, 6.15 μm at the median (50^{th} percentile) and 13.46 μm at the 90^{th} percentile.

### EXAMPLE 3

In this example, the data from entry no. 27 in Table 1 was used. Temalfa type pulp was used to form the slurry from which the microcrystalline cellulose in the form of wet cake was produced. The additive material was colloidal silicon dioxide, which was added to the microcrystalline cellulose in the form of wet cake in an amount of 40.0% weight:weight percent. After spray drying of the suspension, certain particle size resulted, of which the particle size diameter was 4.72 μm at the 10^{th} percentile, 20.23 μm at the median (50^{th} percentile) and 80.76 μm at the 90^{th} percentile. This composition, when dried, produced 1.8% moisture.

### EXAMPLE 4

In this example, the data from entry no. 29 in Table 1 was used. Temalfa type pulp was used to form the slurry from which the microcrystalline cellulose in the form of wet cake was produced. The additive material was colloidal silicon dioxide, which was added to the microcrystalline cellulose in the form of wet cake in an amount of 80.0% weight:weight percent. After spray drying of the suspension, certain particle size resulted, of which the particle size diameter was 2.65 μm at the 10^{th} percentile, 11.55 μm at the median (50^{th} percentile) and 43.80 μm at the 90th percentile. This composition, when dried, produced 2.0% moisture.

### EXAMPLE 5

In this example, the data from entry no. 1 in Table 1 was used. Temalfa type pulp was used to form the slurry from which the microcrystalline cellulose in the form of wet cake was produced. The additive material was ZrO₂, which was added to the microcrystalline cellulose in the form of wet cake in an amount of 2.0% weight:weight percent. After spray drying of the suspension, certain particle size resulted, of which the particle size diameter was 22.36 μm at the 10^{th} percentile, 68.15 μm at the median (50^{th} percentile) and 203.69 μm at the 90^{th} percentile. This composition, when dried, produced 4.9% moisture. The residue on ignition (ROI), a measure of the efficiency of the drying process, was 1.9160%, as compared to the original 2.0%.

### EXAMPLE 6

In this example, the data from entry no. 2 in Table 1 was used. Temalfa type pulp was used to form the slurry from which the microcrystalline cellulose in the form of wet cake was produced. The additive material was Al₂O₃, which was added to the microcrystalline cellulose in the form of wet cake in an amount of 2.0% weight:weight percent. After spray drying of the suspension, certain particle size resulted, of which the particle size diameter was 19.15 μm at the 10^{th} percentile, 58.93 μm at the median (50^{th} percentile) and 1564.19 μm at the 90^{th} percentile. This composition, when dried, produced 3.8% moisture. The residue on ignition (ROI), a measure of the efficiency of the drying process, was 1.9893%, as compared to the original 2.0%.

### EXAMPLE 7

In this example, the data from entry no. 16 in Table 1 was used. Temalfa type pulp was used to form the slurry from which the microcrystalline cellulose in the form of wet cake was produced. The additive material was sodium lauryl sulfate (SLS), which was added to the microcrystalline cellulose in the form of wet cake in an amount of 0.7% weight:weight percent. After spray drying of the suspension, certain particle size resulted, of which the particle size diameter was 11.47 μm at the 10^{th} percentile, 32.93 μm at the median (50^{th} percentile) and 88.79 μm at the 90^{th} percentile. This composition, when dried, produced 3.1% moisture.

### EXAMPLE 8

In this example, the data from entry no. 21 in Table 1 was used. Saicor type pulp was used to form the slurry from which the microcrystalline cellulose in the form of wet cake was produced. The additive material was BaSO₄, which was added to the microcrystalline cellulose in the form of wet cake in an amount of 2.0% weight:weight percent. After spray drying of the suspension, certain particle size resulted, of which the particle size diameter was 12.06 μm at the 10^{th} percentile, 41.02 μm at the median (50^{th} percentile) and 95.75 μm at the 90th percentile. This composition, when dried, produced 5.1% moisture.

### EXAMPLE 9

In this example, the data from entry no. 6 in Table 1 was used. Temalfa type pulp was used to form the slurry from which the microcrystalline cellulose in the form of wet cake was produced. The additive material was PVP, which was added to the microcrystalline cellulose in the form of wet cake in an amount of 5.0% weight:weight percent. A second additive material was colloidal silicon dioxide, which was added to the microcrystalline cellulose in an amount of 5.0% weight:weight percent. After spray drying, certain particle size resulted, of which the particle size diameter was 9.91 μm at the 10^{th} percentile, 31.15 μm at the median (50^{th} percentile) and 104.45 μm at the 90^{th} percentile. This composition, when dried, produced 3.5% moisture.

### EXAMPLE 10

In this example, the data from entry no. 12 in Table 1 was used. Temalfa type pulp was used to form the slurry from which the microcrystalline cellulose in the form of wet cake was produced. The additive material was PVP, which was added to the microcrystalline cellulose in the form of wet cake in an amount of 20.0% weight percent. A second additive material was colloidal silicon dioxide, which was added to the microcrystalline cellulose in an amount of 5.0% weight percent. After spray drying, certain particle size resulted, of which the particle size diameter was 15.961 μm at the 10^{th} percentile, 43.87 μm at the median (50^{th} percentile) and 109.29 μm at the 90^{th} percentile. This composition, when dried, produced 2.9% moisture.

### EXAMPLE 11

In this example, the data from entry no. 23 in Table 1 was used. No pulp was used to form a slurry, and no microcrystalline cellulose in the form of wet cake was produced. The composition was formed of 100.0% colloidal silicon dioxide on a weight percent. After spray drying of the suspension, certain particle size resulted, of which the particle size diameter was 2.04 μm at the 10^{th} percentile, 7.67 μm at the median (50^{th} percentile) and 34.29 μm at the 90^{th} percentile.

### EXAMPLE 12

In this example, the data from entry no. 26 in Table 1 was used. No pulp was used to form a slurry, and no microcrystalline cellulose in the form of wet cake was produced. The composition was formed of 75.0% colloidal silicon dioxide, and a second additive material was SLS, which was added to the colloidal silicon dioxide in an amount of 25.0% weight percent. After spray drying, certain particle size resulted, of which the particle size diameter was 1.89 μm at the 10^{th} percentile, 8.34 μm at the median (50^{th} percentile) and 23.45 μm at the 90^{th} percentile.

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

What is claimed is:

1. A composition comprising a particulate agglomerate of microcrystalline cellulose co-processed with from about 21% to about 99% by weight of an agent selected from the group consisting of silicon dioxide, aluminum silicate, magnesium aluminum silicate and precipitated amorphous silica, the microcrystalline cellulose and agent being in intimate association with each other and said agent being integrated with or at least partially coating said microcrystalline cellulose.

2. The composition of claim 1, wherein said silicon dioxide portion of said agglomerate is derived from colloidal silicon dioxide or fumed silica.

3. The composition of claim 1, wherein said silicon dioxide portion of said agglomerate is derived from a silicon dioxide having a surface area from about 10 m²/g to about 500 m²/g.

4. The composition of claim 1 wherein said composition is used in an industrial application selected from the group consisting of coatings and pigments, cosmetics and sunscreen, insulating additive for wires and cable, ceramics for insulators and computers, heat sinks, spark plugs, colored paper, pet foods and animal feed, paint, adhesives, electroplating, carbon black in cement and mortar, catalytic converters and electronic adhesives.

5. A composition comprising a particulate agglomerate of microcrystalline cellulose co-processed with from about 1% to about 99% by weight of an agent selected from the group consisting of titanium dioxide, aluminum oxide, zirconium dioxide, dibasic calcium phosphate, polyvinyl pyrollidone, carbon black, calcium sulfate and barium sulfate.

6. The composition of claim 5 wherein the microcrystalline cellulose and agent are in intimate association with each other and said agent is integrated with or at least partially coats said microcrystalline cellulose.

7. The composition of claim 5 wherein said composition is used in an industrial application selected from the group consisting of coatings and pigments, cosmetics and sunscreen, insulating additive for wires and cable, ceramics for insulators and computers, heat sinks, spark plugs, colored paper, pet foods and animal feed, paint, adhesives, electroplating, carbon black in cement and mortar, catalytic converters and electronic adhesives.

8. A composition comprising a particulate agglomerate of microcrystalline cellulose co-processed with from about 1% to about 99% by weight of an agent selected from the group consisting of cellulosics, starches, starch derivatives, polysaccharides, polypeptides, polyesters, polyalkanes, polyalkenes, polyalkynes, synthetic resins, natural resins and mixtures thereof.

9. The composition of claim 8 wherein the microcrystalline cellulose and agent are in intimate association with each other and said agent is integrated with or at least partially coats said microcrystalline cellulose.

10. The composition of claim 8 wherein said composition is used in an industrial application selected from the group consisting of coatings and pigments, cosmetics and sunscreen, insulating additive for wires and cable, ceramics for insulators and computers, heat sinks, spark plugs, colored paper, pet foods and animal feed, paint, adhesives, electroplating, carbon black in cement and mortar, catalytic converters and electronic adhesives.

11. A method of preparing a composition, comprising:
a) forming an aqueous slurry containing a mixture of microcrystalline cellulose in the form of wet cake and from about 21% to about 99% by weight of an agent selected from the group consisting of silicon dioxide, aluminum silicate, magnesium aluminum silicate and precipitated amorphous silica; and
b) drying said slurry to obtain a composition comprising a plurality of agglomerated particles of microcrystalline cellulose in intimate association with said agent.

12. The method of claim 11, wherein, when said agent comprises silicon dioxide derived from colloidal silicon dioxide or fumed silica.

13. The method of claim 11, further comprising adding one or more viscosity modifying agents to said slurry prior to said step of drying said slurry.

14. The method of claim 11 further comprising using said composition in an industrial application selected from the group consisting of coatings and pigments, cosmetics and sunscreen, insulating additive for wires and cable, ceramics for insulators and computers, heat sinks, spark plugs, colored paper, pet foods and animal feed, paint, adhesives, electroplating, carbon black in cement and mortar, catalytic converters and electronic adhesives.

15. A method of preparing a composition, comprising:
a) providing an aqueous slurry of microcrystalline cellulose in the form of wet cake;
b) providing an aqueous slurry containing an agent selected from the group consisting of silicon dioxide, aluminum silicate, magnesium aluminum silicate and precipitated amorphous silica; and
c) drying said aqueous slurry of microcrystalline cellulose in the form of wet cake and said aqueous slurry of said agent together in a drying chamber to obtain a composition comprising a plurality of agglomerated particles of microcrystalline cellulose in intimate association with said agent, wherein said particles contain from about 21% to about 99% by weight of said agent.

16. The method of claim 15, wherein said agent comprises silicon dioxide derived from colloidal silicon dioxide or fumed silica.

17. The method of claim 15, further comprising adding one or more viscosity modifying agents prior to said step of drying.

18. The method of claim 15 further comprising using said composition in an industrial application selected from the group consisting of coatings and pigments, cosmetics and sunscreen, insulating additive for wires and cable, ceramics for insulators and computers, heat sinks, spark plugs, colored paper, pet foods and animal feed, paint, adhesives, electroplating, carbon black in cement and mortar, catalytic converters and electronic adhesives.

19. A method of preparing a composition, comprising:
a) providing an aqueous slurry of microcrystalline cellulose in the form of wet cake;
b) providing an agent selected from the group consisting of silicon dioxide, aluminum silicate, magnesium aluminum silicate and precipitated amorphous silica in the form of a dried powder; and
c) drying said aqueous slurry of microcrystalline cellulose in the form of wet cake and said dry powder agent together in a drying chamber to obtain a composition comprising a plurality of agglomerated particles of said microcrystalline cellulose in intimate association with said agent, wherein said particles contain from about 21% to about 99% by weight of said agent.

20. The method of claim 19, wherein said agent comprises silicon dioxide derived from colloidal silicon dioxide or fumed silica.

21. The method of claim 19, further comprising adding one or more viscosity modifying agents prior to said step of drying.

22. The method of claim 19 further comprising using said composition in an industrial application selected from the group consisting of coatings and pigments, cosmetics and sunscreen, insulating additive for wires and cable, ceramics for insulators and computers, heat sinks, spark plugs, colored paper, pet foods and animal feed, paint, adhesives, electroplating, carbon black in cement and mortar, catalytic converters and electronic adhesives.

23. A method of preparing an industrial composition, comprising:
a) forming an aqueous slurry containing a mixture of microcrystalline cellulose in the form of wet cake and an effective amount of an agent selected from the group consisting of titanium dioxide, aluminum oxide, zirconium dioxide, dibasic calcium phosphate, polyvinyl propylene, carbon black, calcium sulfate and barium sulfate; and
b) drying said slurry to obtain a composition comprising a plurality of agglomerated particles of microcrystalline cellulose in intimate association with said agent.

24. The method of claim 23, wherein said agent is mixed with said microcrystalline cellulose in an amount from about 1% to about 99% by weight.

25. The method of claim 23, further comprising adding one or more viscosity modifying agents to said slurry prior to said step of drying said slurry.

26. The method of claim 23 further comprising using said composition in an industrial application selected from the group consisting of coatings and pigments, cosmetics and sunscreen, insulating additive for wires and cable, ceramics for insulators and computers, heat sinks, spark plugs, colored paper, pet foods and animal feed, paint, adhesives, electroplating, carbon black in cement and mortar, catalytic converters and electronic adhesives.

27. A method of preparing a composition, comprising:
a) providing an aqueous slurry of microcrystalline cellulose in the form of wet cake;
b) providing an aqueous slurry containing an agent selected from the group consisting of titanium dioxide, aluminum oxide, zirconium dioxide, dibasic calcium phosphate, polyvinyl propylene, carbon black, calcium sulfate and barium sulfate; and
c) drying said aqueous slurry of microcrystalline cellulose in the form of wet cake and said aqueous slurry of said agent together in a drying chamber to obtain a composition comprising a plurality of agglomerated particles of microcrystalline cellulose in intimate association with said agent.

28. The method of claim 27, wherein said particles contain from about 1% to about 99% by weight of said agent.

29. The method of claim 27, further comprising adding one or more viscosity modifying agents prior to said step of drying.

30. The method of claim 27 further comprising using said composition in an industrial application selected from the group consisting of coatings and pigments, cosmetics and sunscreen, insulating additive for wire and cable, ceramics for insulators and computers, heat sinks, spark plugs, colored paper, pet foods and animal feed, paint, adhesives, electroplating, carbon black in cement and mortar, catalytic converters and electronic adhesives.

31. A method of preparing a composition, comprising:
a) providing an aqueous slurry of microcrystalline cellulose in the form of wet cake;
b) providing an agent selected from the group consisting of titanium dioxide, aluminum oxide, zirconium dioxide, dibasic calcium phosphate, polyvinyl propylene, carbon black, calcium sulfate and barium sulfate; and
c) drying said aqueous slurry of microcrystalline cellulose in the form of wet cake and said dry powder agent together in a drying chamber to obtain a composition comprising a plurality of agglomerated particles of microcrystalline cellulose in intimate association with said agent.

32. The method of claim 31, wherein said particles contain from about 1% to about 99% by weight of said agent.

33. The method of claim 31, further comprising adding one or more viscosity modifying agents prior to said step of drying.

34. The method of claim 31 further comprising using said composition in an industrial application selected from the group consisting of coatings and pigments, cosmetics and sunscreen, insulating additive for wire and cable, ceramics for insulators and computers, heat sinks, spark plugs, colored paper, pet foods and animal feed, paint, adhesives, electroplating, carbon black in cement and mortar, catalytic converters and electronic adhesives.

35. A method of preparing an industrial composition, comprising:
a) forming an aqueous slurry containing a mixture of microcrystalline cellulose in the form of wet cake and an effective amount of an agent selected from the group consisting of celluloses, starches, starch derivatives, polysaccharides, polypeptides, polyesters, polyalkanes, alkenes, alkynes, synthetic resins, natural resins and mixtures thereof; and
b) drying said slurry to obtain a composition comprising a plurality of agglomerated particles of microcrystalline cellulose in intimate association with said agent.

36. The method of claim 35, wherein said agent is mixed with said microcrystalline cellulose in an amount from about 1% to about 99% by weight.

37. The method of claim 35, further comprising adding one or more viscosity modifying agents to said slurry prior to said step of drying said slurry.

38. The method of claim 35 further comprising using said composition in an industrial application selected from the group consisting of coatings and pigments, cosmetics and sunscreen, insulating additive for wire and cable, ceramics for insulators and computers, heat sinks, spark plugs, colored paper, pet foods and animal feed, paint, adhesives, electroplating, carbon black in cement and mortar, catalytic converters and electronic adhesives.

39. A method of preparing a composition, comprising:
a) providing an aqueous slurry of microcrystalline cellulose in the form of wet cake;
b) providing an aqueous slurry containing an agent selected from the group consisting of celluloses, starches, starch derivatives, polysaccharides, polypeptides, polyesters, polyalkanes, alkenes, alkynes, synthetic resins, natural resins and mixtures thereof; and
c) drying said aqueous slurry of microcrystalline cellulose in the form of wet cake and said aqueous slurry of said agent together in a drying chamber to obtain a composition comprising a plurality of agglomerated particles of microcrystalline cellulose in intimate association with said agent.

40. The method of claim 39, wherein said particles contain from about 1% to about 99% by weight of said agent.

41. The method of claim 39, further comprising adding one or more viscosity modifying agents prior to said step of drying.

42. The method of claim 39 further comprising using said composition in an industrial application selected from the group consisting of coatings and pigments, cosmetics and sunscreen, insulating additive for wire and cable, ceramics for insulators and computers, heat sinks, spark plugs, colored paper, pet foods and animal feed, paint, adhesives, electroplating, carbon black in cement and mortar, catalytic converters and electronic adhesives.

43. A method of preparing a composition, comprising:
a) providing an aqueous slurry of microcrystalline cellulose in the form of wet cake;
b) providing an agent selected from the group consisting of celluloses, starches, starch derivatives, polysaccharides, polypeptides, polyesters, polyalkanes, alkenes, alkynes, synthetic resins, natural resins and mixtures thereof; and
c) drying said aqueous slurry of microcrystalline cellulose and said dry powder agent together in a drying chamber to obtain a composition comprising a plurality of agglomerated particles of microcrystalline cellulose in intimate association with said agent.

44. The method of claim 43, wherein said particles contain from about 1% to about 99% by weight of said agent.

45. The method of claim 43, further comprising adding one or more viscosity modifying agents prior to said step of drying.

46. The method of claim 43 further comprising using said composition in an industrial application selected from the group consisting of coatings and pigments, cosmetics and sunscreen, insulating additive for wire and cable, ceramics for insulators and computers, heat sinks, spark plugs, colored paper, pet foods and animal feed, paint, adhesives, electroplating, carbon black in cement and mortar, catalytic converters and electronic adhesives.

47. The composition of claim 1, wherein the composition has a median particle size from about 5 μm to about 350 μm.

48. The composition of claim 1, wherein the composition has a median particle size from about 65 μm to about 350 μm.

49. The composition of claim 1, wherein the composition has a median particle size from about 120 μm to about 200 μm.

50. The composition of claim 5, wherein the composition has a median particle size from about 65 μm to about 350 μm.

51. The composition of claim 8, wherein the composition has a median particle size from about 65 μm to about 350 μm.

52. The method of claim 11, wherein the composition has a median particle size from about 5 μm to about 350 μm.

53. The method of claim 11, wherein the composition has a median particle size from about 65 μm to about 350 μm.

54. The method of claim 11, wherein the composition has a median particle size from about 120 μm to about 200 μm.

55. The method of claim 15, wherein the composition has a median particle size from about 65 μm to about 350 μm.

56. The method of claim 19, wherein the composition has a median particle size from about 65 μm to about 350 μm.

57. The method of claim 23, wherein the composition has a median particle size from about 65 μm to about 350 μm.

58. The method of claim 27, wherein the composition has a median particle size from about 65 μm to about 350 μm.

59. The method of claim 31, wherein the composition has a median particle size from about 65 μm to about 350 μm.

60. The method of claim 35, wherein the composition has a median particle size from about 65 μm to about 350 μm.

61. The method of claim 39, wherein the composition has a median particle size from about 65 μm to about 350 μm.

62. The method of claim 43, wherein the composition has a median particle size from about 65 μm to about 350 μm.
